(19) United States
(12) Patent Application Publication
(10) Pub. No.: US 2004/0097817 A1
(43) Pub. Date: May 20, 2004

(54) Superior-limb arteriostenosis evaluating apparatus

(75) Inventor: Tsuneo Nakagawa, Komaki-shi (JP)

Correspondence Address: OLIFF & BERRIDGE, PLC, P.O. BOX 19928, ALEXANDRIA, VA, 22320, US

(73) Assignee: COLIN CORPORATION, Komaki-shi (JP)

(21) Appl. No.: 10/693,523

(22) Filed: Oct. 27, 2003

(30) Foreign Application Priority Data
Nov. 18, 2002 (JP) 2002-334318

(51) Int. Cl. A61B 5/02

(52) U.S. Cl. 600/500

(57) ABSTRACT

A superior-limb arteriostenosis evaluating apparatus, including a superior-limb pulse-wave detecting device which detects a superior-limb pulse wave produced from an artery of at least one of a left superior limb and a right superior limb of a living subject, an inferior-limb pulse-wave detecting device which detects an inferior-limb pulse wave produced from an artery of at least one of a left inferior limb and a right inferior limb of the subject, and an arteriostenosis judging device for judging whether the artery of at least one of the left and right superior limbs has an arteriostenosis, based on a phase difference between the superior-limb pulse wave detected by the superior-limb pulse-wave detecting device and the inferior-limb pulse wave detected by the inferior-limb pulse-wave detecting device.

### BACKGROUND OF THE INVENTION

1. Field of the Invention

The present invention relates to a superior-limb arteriostenosis evaluating apparatus that can evaluate stenosis of an artery of a superior limb of a living being, the super-limb artery including a subclavian artery. In the present specification, the superior-limb artery is defined as including the subclavian artery, and an axillary artery, a brachial artery, an ulnar artery, and a radial artery that communicate with the subclavian artery.

2. Related Art Statement

Atherosclerosis is a sort of arteriosclerosis that is characterized in that lipid, in particular, cholesterol deposits on inner surfaces of arterial walls and accordingly the arterial walls thicken. Since atherosclerosis causes stenosis of blood vessels and accordingly decreases diameters of the blood vessels, it is also called arteriostenosis or arteriosclerosis obliterans.

There is known an ankle-and-brachium-blood-pressure-index measuring device that inspects arteriostenosis by utilizing a fact that if an artery has stenosis, then blood pressure lowers on a downstream side of the stenotic portion of the artery. An example of the index measuring device is disclosed by Patent Document 1 (i.e., Japanese Patent No. 3140007 or its corresponding U.S. Pat. No. 6,355,000). The disclosed index measuring device includes respective cuffs that are wound on a brachium and an ankle of a living being so as to measure a brachium blood pressure and an ankle blood pressure, calculates an ankle-and-brachium blood pressure index as a ratio between the measured ankle and brachium blood pressure values, and inspects whether the subject has arteriostenosis based on the thus obtained ankle-and-brachium blood pressure index.

Generally, an ankle-and-brachium blood pressure index is calculated, using a systolic ankle blood pressure and a systolic brachium blood pressure, as a proportion of the ankle systolic blood pressure relative to the brachium systolic blood pressure. In this case, if the ankle-and-brachium blood pressure index is smaller than 0.9, that is, if the ankle systolic blood pressure is smaller than a product of the brachium systolic blood pressure and 0.9, it is judged that the person is suspected to have arteriostenosis.

In many cases, arteriostenosis occurs to arteries of inferior limbs. However, there are some cases where arteries of superior limbs suffer stenosis. For example, there is known subclavian arteriostenosis in which subclavian artery suffers stenosis. However, since ankle-and-brachium blood pressure index is the proportion of ankle blood pressure relative to brachium blood pressure, as explained above, the index is obtained on the assumption that stenosis is more likely to occur to inferior-limb arteries. More specifically explained, the index is for comparing ankle blood pressure and brachium blood pressure with each other, judging whether the ankle blood pressure is abnormal, and judging whether an artery between the heart and the ankle, in particular, an inferior-limb artery has stenosis. Thus, it has been difficult to make a diagnosis on whether a superior-limb artery has stenosis, based on ankle-and-brachium blood-pressure index.

### SUMMARY OF THE INVENTION

It is therefore an object of the present invention to provide a superior-limb arteriostenosis evaluating apparatus which can accurately evaluate stenosis of an artery of a superior limb of a living being.

The above object has been achieved by the present invention. According to the present invention, there is provided a superior-limb arteriostenosis evaluating apparatus, comprising a superior-limb pulse-wave detecting device which detects a superior-limb pulse wave produced from an artery of at least one of a left superior limb and a right superior limb of a living subject; an inferior-limb pulse-wave detecting device which detects an inferior-limb pulse wave produced from an artery of at least one of a left inferior limb and a right inferior limb of the subject; and an arteriostenosis judging means for judging whether the artery of the at least one of the left and right superior limbs has an arteriostenosis, based on a phase difference between the superior-limb pulse wave detected by the superior-limb pulse-wave detecting device and the inferior-limb pulse wave detected by the inferior-limb pulse-wave detecting device.

According to the present invention, the superior-limb pulse-wave detecting device detects the superior-limb pulse wave produced from the artery of at least one of the left and right superior limbs of the living subject, the inferior-limb pulse-wave detecting device detects the inferior-limb pulse wave produced from the artery of at least one of the left and right inferior limbs of the subject, and the arteriostenosis judging means judges whether the artery of the at least one of the left and right superior limbs has stenosis, based on the phase difference between the detected superior-limb pulse wave and the detected inferior-limb pulse wave. Therefore, the present apparatus can accurately evaluate the stenosis of the superior-limb artery.

Here, preferably, the arteriostenosis judging means comprises a phase-difference calculating means for calculating the phase-difference between the superior-limb pulse wave detected by the superior-limb pulse-wave detecting device and the inferior-limb pulse wave detected by the inferior-limb pulse-wave detecting device; and a phase-difference judging means for judging whether the phase difference calculated by the phase-difference calculating means is smaller than a reference value, and judging that the artery of the at least one of the left and right superior limbs has the arteriostenosis, when it is judged that the phase difference calculated by the phase-difference calculating means is smaller than the reference value. According to this feature, if the phase difference between the superior-limb pulse wave and the inferior-limb pulse wave is smaller than the reference value, it is judged that the artery of the superior limb has stenosis. Thus, the stenosis of the superior-limb artery can be accurately evaluated.

Also, preferably, the inferior-limb pulse-wave detecting device detects a left-inferior-limb pulse wave produced from an artery of the left inferior limb and a right-inferior-limb pulse wave produced from an artery of the right inferior limb, the arteriostenosis judging means comprises a pulse-wave selecting means for selecting one of the left-inferior-limb pulse wave and the right-inferior-limb pulse wave that has a faster phase than a phase of the other inferior-limb pulse wave; and means for judging whether the artery of the at least one of the left and right superior limbs has the arteriostenosis, based on the phase difference between the superior-limb pulse wave detected by the superior-limb pulse-wave detecting device and the one inferior-limb pulse wave selected by the pulse-wave selecting means. According to this feature, it is judged whether the artery of at least one of the left and right superior limbs has stenosis, based on the phase difference between the superior-limb pulse wave detected from at least one of the left and right superior limbs and one of the left-inferior-limb and right-inferior-limb pulse waves that has the faster phase. Thus, the stenosis of the superior-limb artery can be accurately evaluated. If it is assumed that both the left-inferior-limb and right-inferior-limb pulse waves can be influenced by stenosis, then it can be said that one of the left-inferior-limb and right-inferior-limb pulse waves that has the faster phase is less influenced by stenosis than the other inferior-limb pulse wave. This is the reason why the stenosis of the superior-limb artery can be accurately evaluated.

Also, preferably, the arteriostenosis judging means judges whether the artery of the at least one of the left and right superior-limbs has the arteriostenosis, based on the phase difference between a rising point of the superior-limb pulse wave detected by the superior-limb pulse-wave detecting device and a rising point of the inferior-limb pulse wave detected by the inferior-limb pulse-wave detecting device. According to this feature, it is judged whether the artery of at least one of the left and right superior limbs has stenosis, based on the phase difference between the rising point of the superior-limb pulse wave and the rising point of the inferior-limb pulse wave. Thus, the stenosis of the superior-limb artery can be accurately evaluated. A rising point of a pulse wave is less influenced by a reflected wave than a peak point of the pulse wave. This is the reason why the stenosis of the superior-limb artery can be accurately evaluated.

Also, preferably, the superior-limb arteriostenosis evaluating apparatus further comprises an indication outputting means for outputting, when the arteriostenosis judging means judges that the artery of the at least one of the left and right superior limbs has the arteriostenosis, an indication indicating that the artery of the at least one of the left and right superior limbs has the arteriostenosis. According to this feature, the indication outputting means outputs the indication indicating that the artery of at least one of the left and right superior limbs has stenosis. Thus, a person such as the subject or a doctor can clearly know the stenosis of the superior-limb artery.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a diagrammatic view for explaining essential portions of a circuitry of a superior-limb arteriostenosis evaluating apparatus to which the present invention is applied;

FIG. 2 is a diagrammatic view for explaining essential control functions of an electronic control device shown in FIG. 1;

FIG. 3 is a view for explaining respective positions of cuffs shown in FIG. 1 relative to respective arteries A, B, C, D, E, F of a living subject;

FIG. 4 is a graph for explaining a phase difference between each of a left-brachium pulse wave and a right-brachium pulse wave, and an ankle pulse wave; and

FIG. 5 is a flow chart for explaining the essential control functions of the electronic control device shown in FIG. 1.

### DETAILED DESCRIPTION OF PREFERRED EMBODIMENTS

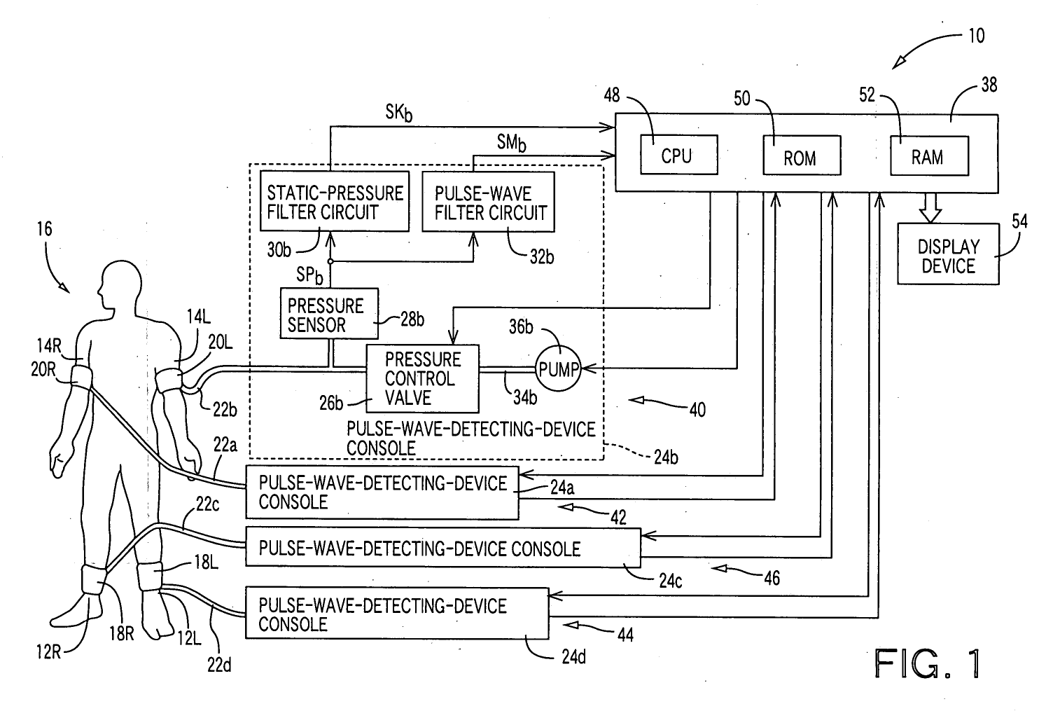

Hereinafter, there will be described an embodiment of the present invention in detail by reference to the drawings. FIG. 1 is a diagrammatic view for explaining a circuitry of an arteriosclerosis evaluating apparatus 10 to which the present invention is applied.

In FIG. 1, the arteriosclerosis evaluating apparatus 10 includes a left-ankle cuff 18L and a right-ankle cuff 18R that are wound around a left ankle 12L and a right ankle 12R, respectively, of a patient 16 as a living subject; and a left-brachium cuff 20L and a right-brachium cuff 20R that are wound around a left brachium 14L and a right brachium 14R, respectively, of the patient 16. Each of the cuffs 18L, 18R, 20L, 20R provides a pressing band that presses a body portion of the living subject on which the each cuff is worn, and includes a belt-like outer bag that is formed of a non-stretchable material, such as cloth or polyester, and an inflatable rubber bag accommodated in the outer bag.

The left and right brachium cuffs 20L, 20R are connected via respective pipes 22b, 22a to respective consoles 24b, 24a of respective pulse-wave detecting devices; and the left and right ankle cuffs 18L, 18R are connected via respective pipes 22d, 22c to respective consoles 24d, 24c of respective pulse-wave detecting devices.

Since the respective consoles 24a, 24b, 24c, 24d of the four pulse-wave detecting devices have an identical construction, the basic construction of the pulse-wave detecting-device console 24b connected to the left brachium cuff 20L will be described below as a representative of the four consoles 24. The pulse-wave-detecting-device console 24b includes a pressure control valve 26b, a pressure sensor 28b, a static-pressure filter circuit 30b, a pulse-wave filter circuit 32b, a pipe 34b, and an air pump 36b, and the pipe 22b is connected to the pressure sensor 28b and the pressure control valve 26b which in turn is connected to the air pump 36b, via the pipe 34b.

The pressure control valve 26b adjusts a pressure of a pressurized air supplied from the air pump 36b, and supplies the pressure-adjusted air to the left brachium cuff 20L so as to control an air pressure in the cuff 20L, i.e., a pressing pressure applied to the living subject, or discharges the air from the cuff 20L so as to release the pressing of the cuff 20L.

The pressure sensor 28b detects the air pressure in the left brachium cuff 20L, and supplies a pressure signal, SPb, representing the detected air pressure, to the static-pressure filter circuit 30b and the pulse-wave filter circuit 32b. The static-pressure filter circuit 30b includes a low-pass filter that permits passing of a signal having a low frequency not higher than 1 Hz and thereby separates (i.e., extracts), from the pressure signal SPb, a left-brachium-cuff-pressure signal, SKb, representing a static or direct-current component of the detected air pressure, i.e., a pressing pressure of the cuff 20L (hereinafter, referred to as the left-brachium-cuff pressure, PCb). The filter circuit 30b supplies the left-brachium-cuff-pressure signal SKb to an electronic control device 38 via an AID (analog-to-digital) converter, not shown.

The pulse-wave filter circuit 32b includes a bandpass filter that permits passing of a sound signal having a frequency of from 1 Hz to ten and several Hz and thereby separates (extracts), from the pressure signal SPb, a left-brachium-pulse-wave signal, SMb, representing an oscillatory component of the detected air pressure that is produced in synchronism with heartbeats of the living subject and is mixed with the air pressure. The filter circuit 32b supplies the left-brachium-pulse-wave signal SMb to the electronic control device 38 via an A/D converter, not shown. The left-brachium-pulse-wave signal SMb represents a left-brachium pulse wave, WB_{L}, that is produced from an artery of the left brachium 14L being pressed by the left brachium cuff 20L. Thus, the left brachium cuff 20L and the pulse-wave-detecting-device console 24b cooperate with each other to provide a left-brachium-pulse-wave detecting device 40.

Likewise, a right-brachium-pulse-wave signal, SMa, separated by a pulse-wave filter circuit 32a of the pulse-wave-detecting-device console 24a represents a right-brachium pulse wave, WB_{R}, that is produced from an artery of the right brachium 14R being pressed by the right brachium cuff 20R. Thus, the right brachium cuff 20R and the pulse-wave-detecting-device console 24a cooperate with each other to provide a right-brachium-pulse-wave detecting device 42. Also, a left-ankle-pulse-wave signal, SMd, separated by a pulse-wave filter circuit 32d of the pulse-wave-detecting-device console 24d represents a left-ankle pulse wave, WA_{L}, that is produced from an artery of the left ankle 12L being pressed by the left-ankle cuff 18L. Thus, the left ankle cuff 18L and the pulse-wave-detecting-device console 24d cooperate with each other to provide a left-ankle-pulse-wave detecting device 44. Also, a right-ankle-pulse-wave signal, SMc, separated by a pulse-wave filter circuit 32c of the pulse-wave-detecting-device console 24c represents a right-ankle pulse wave, WA_{R}, that is produced from an artery of the right ankle 12R being pressed by the right-ankle cuff 18R. Thus, the right ankle cuff 18R and the pulse-wave-detecting-device console 24c cooperate with each other to provide a right-ankle-pulse-wave detecting device 46. Each of the left-brachium and right-brachium pulse-wave detecting devices 40, 42 provides a superior-limb pulse-wave detecting device; and each of the left-ankle and right-ankle pulse-wave detecting devices 44, 46 provides an inferior-limb pulse-wave detecting device.

The electronic control device 38 is provided by a so-called microcomputer including a CPU (central processing unit) 48, a ROM (read only memory) 50, a RAM (random access memory) 52, and an I/O (input-and-output) port, not shown. The CPU 48 processes signals according to the control programs pre-stored in the ROM 50 by utilizing the temporary-storage function of the RAM 52, and supplies drive signals via the I/O port to the air pump 36 and the pressure control valve 26 of each pulse-wave-detecting-device console 24, so as to control the air pressure in a corresponding one of the cuffs 18, 20. In addition, the CPU 48 processes the signals supplied to the control device 38, so as to measure respective blood-pressure values of the respective body portions on which the four cuffs 18, 20 are worn according to an oscillometric method, and detect respective cuff pulse waves as respective pressure oscillations occurring to the four cuffs 18, 20, and operates a display device 54 to display the thus measured blood-pressure values and cuff pulse waves. In addition, the CPU 48 evaluates stenosis of a superior-limb artery based on a phase difference obtained from the four cuff pulse waves, and operates the display device 54 to display the evaluation results. Moreover, the CPU 48 calculates a superior-and-inferior-limb blood-pressure ratio based on the measured blood-pressure values, and operates the display device 54 to display the thus calculated superior-and-inferior-limb blood-pressure ratio. In addition, based on the evaluation results, the CPU 48 identifies a stenotic artery of the living subject and operates the display device 54 to display the thus identified stenotic artery. The display device 54 may be provided by an image indicator that has an image screen defined by a matrix of picture elements the lightness or chromaticity of each of which is optically changeable and indicates an image on the screen; or a printer that indicates an image on a sheet of paper.

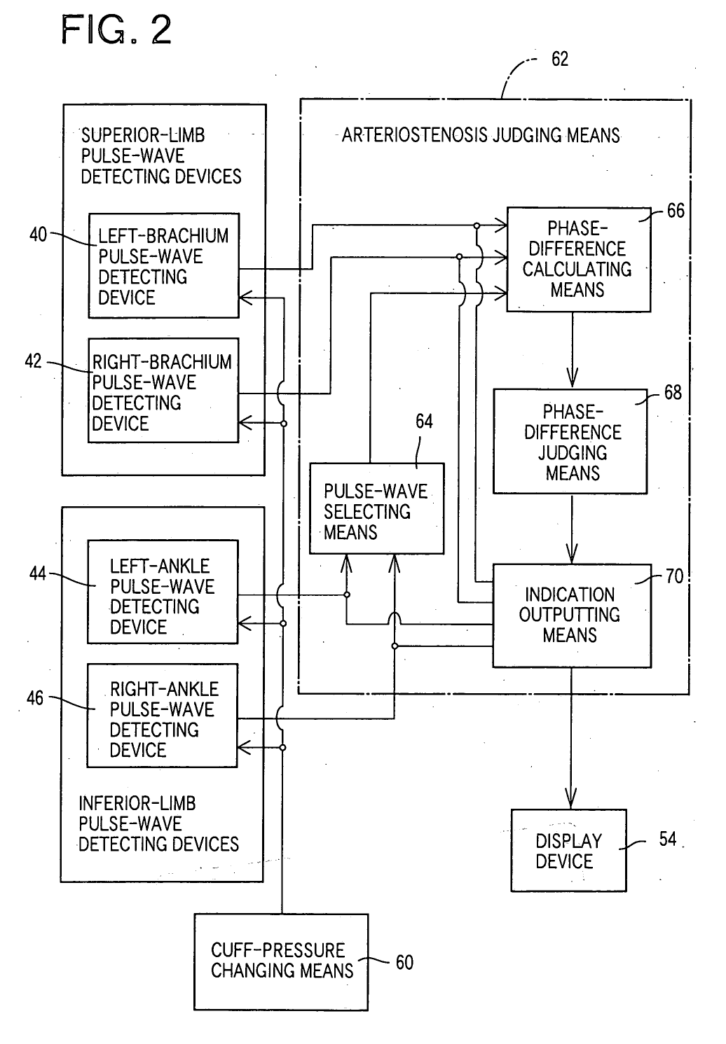

FIG. 2 is a diagrammatic view for explaining essential control functions of the electronic control device 38, that is, a superior-limb-arteriostenosis evaluating function of the control device 38. A cuff-pressure changing device or means 60 operates the respective pressure control valves 26a, 26b, 26c, 26d and the respective air pumps 36a, 36b, 36c, 36d of the pulse-wave detecting devices 40, 42, 44, 46, so as to change each of the respective cuff pressures PCa, PCb, PCc, PCd to a pre-set pulse-wave detection pressure. The pulse-wave detection pressure is pre-set at a pressure that is lower than a mean blood pressure of the body portion on which the cuff 18, 20 is worn, preferably lower than a diastolic blood pressure of the body portion, and assures that each of the pulse-wave signals SM extracted by the pulse-wave filter circuits 32 is not distorted and has a sufficiently great magnitude, for example, a pressure within a range of from 30 mmHg to 60 mmHg.

An arteriostenosis judging device or means 62 judges stenosis of a superior-limb artery of the living subject 16 based on a phase difference between each of the left-brachium and right-brachium pulse waves WB_{L}, WB_{R} detected by the brachium (superior-limb) pulse wave detecting devices 40, 42 and one of the left-ankle and right-ankle pulse waves WA_{L}, WA_{R} detected by the ankle (inferior-limb) pulse wave detecting devices 44, 46. For example, the arteriostenosis judging means 62 selects one of the left-ankle and right-ankle pulse waves WA_{L}, WA_{R} detected by the ankle pulse wave detecting devices 44, 46 from the left and right inferior limbs, such that the selected one ankle pulse wave WA has a faster phase than that of the other ankle pulse wave WA; then calculates a phase difference, DT_{L}, DT_{R}, between each of the left-brachium and right-brachium pulse waves WB_{L}, WB_{R} detected by the brachium (superior-limb) pulse wave detecting devices 40, 42 and the selected one ankle pulse wave WA; and judges stenosis of each of the left-superior-limb artery and the right-superior-limb artery of the living subject 16.

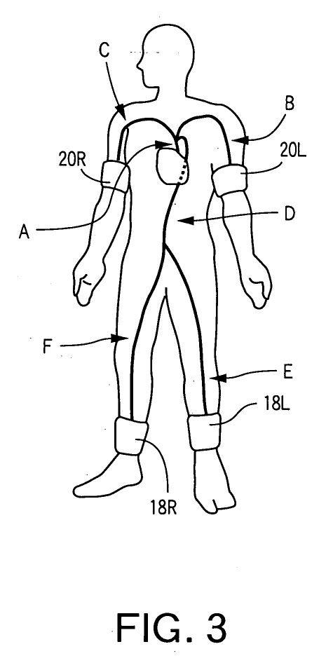

FIG. 3 shows arteries A, B, C, D, E, F of a human being. The artery A starts with the heart and ends with a bifurcation (hereinafter, referred to as the first bifurcation) with which the arteries B, C start; the artery B starts with the first bifurcation and ends with the body portion on which the left brachium cuff 20L is worn; the artery C starts with the first bifurcation and ends with the body portion on which the right brachium cuff 20R is worn; the artery D starts with the first bifurcation and ends with another bifurcation (hereinafter, referred to as the second bifurcation) with which the arteries E, F start; the artery E starts with the second bifurcation and ends with the body portion on which the left ankle cuff 18L is worn; and the artery F starts with the second bifurcation and ends with the body portion on which the right ankle cuff 18R is worn. Thus, the left-superior-limb artery including the left subclavian artery, the left axilliary artery, and the left brachial artery corresponds to the artery B; and the right-superior-limb artery including the right subclavian artery, the right axilliary artery, and the right brachial artery corresponds to the artery C.

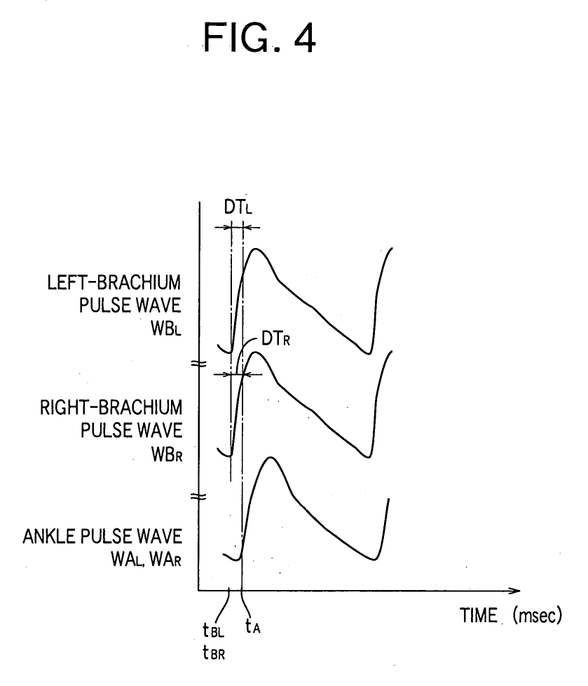

The arteriostenosis judging device or means 62 includes, e.g., a pulse-wave selecting device or means 64, a phase-difference calculating device or means 66, a phase-difference judging device or means 68, and an indication outputting device or means 70. The pulse-wave selecting means 64 selects one of the left-ankle and right-ankle pulse waves WA_{L}, WA_{R} detected by the ankle pulse wave detecting devices 44, 46, such that the selected one ankle pulse wave WA has a faster phase than that of the other ankle pulse wave. WA. The phase-difference calculating means 66 calculates a phase difference DT_{L} between the left-brachium pulse wave WB_{L} detected by the left-brachium pulse wave detecting device 40 and the selected one ankle pulse-wave WA, and a phase difference DT_{R} between the right-brachium pulse wave WB_{R} detected by the right-brachium pulse wave detecting device 42 and the selected one ankle pulse wave WA. As shown in FIG. 4, the phase difference DT_{L} is, e.g., a time difference (msec) between a rising point, t_{BL}, of the left-brachium pulse waves WB_{L} and a rising point, t_{A}, of the ankle pulse wave WA selected by the pulse-wave selecting means 64, and the phase difference DT_{R} is, e.g., a time difference (msec) between a rising point, t_{BR}, of the right-brachium pulse wave WB_{R} and the rising point t_{A} of the selected ankle pulse wave WA, and accordingly the two phase differences DT_{L}, DT_{R} are expressed by the following Equations (1), (2), respectively:

DT_{L}=(t_{A}−t_{BL}) (1)

DT_{R}=(t_{A}−t_{BL}) (2)

The phase-difference judging means 68 judges stenosis of each of the left-superior-limb artery and the right-superior-limb artery of the living subject 16, by judging whether a corresponding one of the phase differences DT_{L}, DT_{R} calculated by the phase-difference calculating means 66 is smaller than a pre-set reference value, t_{J}. The reference value t_{J} is clinically obtained in advance as a value with which it is possible to judge whether the left-brachium pulse wave WB_{L} or the right-brachium pulse wave WB_{R} is delayed by arteriostenosis occurring to the left left-superior-limb artery or the right-superior-limb artery. The reference value t_{J} is, e.g., a positive value near to zero. For example, if the rising point t_{BL} of the left-brachium pulse wave WB_{L} or the rising point t_{BR} of the right-brachium pulse wave WB_{R} is detected after the rising point t_{A} of the ankle pulse wave WA and accordingly the phase difference DT_{L} or DT_{R} is a negative value, the phase-difference judging means 68 judges that the phase difference DT_{L}, DT_{R} is lower than the reference value t_{J}. The indication outputting means 70 operates the display device 54 to display, as shown in FIG. 4, the left-brachium pulse wave WB_{L}, the right-brachium pulse wave WB_{R}, and the selected one of the left and right ankle pulse waves WA_{L}, WA_{R} that has the faster phase, along a common axis indicative of time, such that the three pulse waves are comparable with each other. In addition, the indication outputting means 70 operates the display device 54 to display a symbol or a message, or flash a lamp, to indicate a possibility that the left and/or right superior-limb arteries for which the phase-difference judging means 68 has judged that the phase differences DT_{L} and/or DT_{R} are lower than the reference value t_{J} have stenosis. For example, if the phase difference DT_{L} has been judged as being lower than the reference value t_{J}, then the indication outputting means 70 operates the display device 54 to display an indication that the left superior-limb artery or the left subclavian artery may have stenosis.

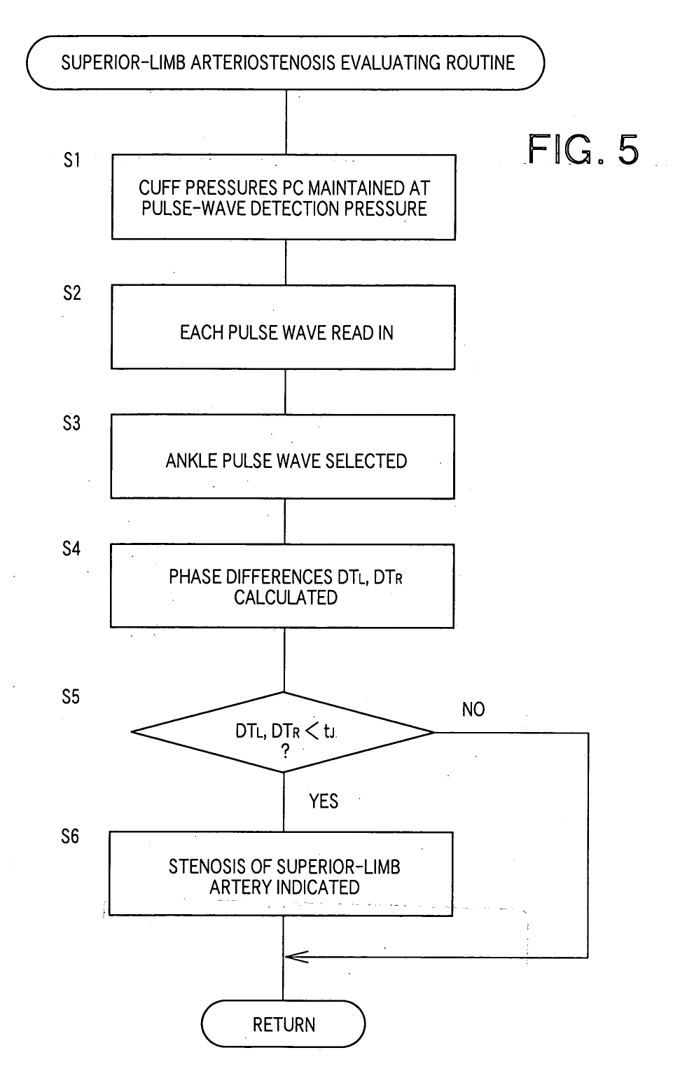

FIG. 5 is a flow chart representing the essential control functions of the electronic control device 38, i.e., the superior-limb arteriostenosis evaluating routine. The flow chart is carried out after respective blood-pressure values of the respective body portions on which the left brachium cuff 20L, the right brachium cuff 20R, the left ankle cuff 18L, and the right ankle cuff 18R are worn are measured to determine an ABI value (i.e., a superior-and-inferior-limb blood-pressure ratio).

In FIG. 5, first, the control device carries out Step S1 (hereinafter, the term “Step” is omitted) corresponding to the cuff-pressure changing means 60. At S1, the control device operates the air pumps 36a, 36b, 36c, 36d and the pressure control valves 26a, 26b, 26c, 26d so as to change and maintain each of the cuff pressures PCa, PCb, PCc, PCd to and at the previously-described pulse-wave detection pressure. Then, the control proceeds with S2 to read in the left-ankle and right-ankle pulse waves WA_{L}, WA_{R} respectively detected by the left-ankle and right-ankle pulse wave detecting devices 44, 46 and the left-brachium and right-brachium pulse waves WB_{L}, WB_{R} respectively detected by the left-brachium and right-brachium pulse wave detecting devices 40, 42.

Then, the control goes to S3 corresponding to the pulse-wave selecting means 64. At S3, the control device selects one of the left-ankle and right-ankle pulse waves WA_{L}, WA_{R} respectively detected by the left-ankle and right-ankle pulse wave detecting devices 44, 46, such the selected one ankle pulse wave WA has the faster phase than that of the other ankle pulse wave WA. Subsequently, the control goes to S4 corresponding to the phase-difference calculating means 66. At S4, the control device calculates a phase difference DT_{L} between the ankle pulse wave WA selected at S3 and the left-brachium pulse wave WB_{L} detected by the left-brachium pulse wave detecting device 40, and a phase difference DT_{R} between the ankle pulse wave WA selected at S3 and the right-brachium pulse wave WB_{R} detected by the right-brachium pulse wave detecting device 42, based on a rising point t_{A} of the selected ankle pulse wave WA, a rising point t_{BL} of the left-brachium pulse waves WB_{L}, and a rising point t_{BR} of the right-brachium pulse wave WB_{R}, according to Equations (1), (2).

Then, the control goes to S5 corresponding to the phase-difference judging means 68. At S5, the control device judges stenosis of each of the left-superior-limb artery and the right-superior-limb artery of the living subject 16, by judging whether a corresponding one of the phase differences DT_{L}, DT_{R} calculated at S4 is smaller than a pre-set reference value t_{J}. Then, the control goes to S6 corresponding to the indication outputting means 70. At S6, the control device operates the display device 54 to display, as shown in FIG. 4, the left-brachium pulse wave WB_{L}, the right-brachium pulse wave WB_{R}, and the selected one of the left and right ankle pulse waves WA_{L}, WA_{R} that has the faster phase, along a common axis indicative of time, such that the three pulse waves are comparable with each other. In addition, the control device operates the display device 54 to display a symbol or a message, or flash a lamp, to indicate a possibility that the left and/or right superior-limb arteries for which the phase-difference judging means 68 has judged that the phase differences DT_{L} and/or DT_{R} are lower than the reference value t_{J} have stenosis.

In the illustrated embodiment, the brachium-pulse-wave detecting devices 40, 42 each functioning as the superior-limb pulse-wave detecting device detect the left-brachium pulse wave WB_{L} and the right-brachium pulse wave. WB_{R} (i.e., the superior-limb pulse waves) produced from the respective arteries of the left and right superior limbs of the living subject, and the ankle-pulse-wave detecting devices 44, 46 each functioning as the inferior-limb pulse-wave detecting device detect the left-ankle pulse wave WA_{L} and the right-ankle pulse wave WA_{R} (i.e., the inferior-limb pulse waves) produced from the respective arteries of the left and right inferior limbs of the subject. The arteriostenosis judging means 62 (S3, S4, S5, S6) judges whether the artery of each of the left and right superior limbs has stenosis, based on the phase difference DT_{L}, DT_{R} between a corresponding of the left-brachium pulse wave WB_{L} and the right-brachium pulse wave WB_{R} and one of the left-ankle pulse wave WA_{L} and the right-ankle pulse wave WA_{R}. Therefore, the present apparatus can accurately evaluate the stenosis of the superior-limb artery.

Also, in the illustrated embodiment, the arteriostenosis judging means 62 includes the phase-difference calculating means 66 (S4) that calculates the phase difference DT_{L}, DT_{R} between each of the left-brachium pulse wave WB_{L} and the right-brachium pulse wave WB_{R} produced by the respective arteries of the left and right superior limbs and detected by the brachium-pulse-wave detecting devices 40, 42 each functioning as the superior-limb pulse-wave detecting device, and one of the left-ankle pulse wave WA_{L} and the right-ankle pulse wave WA_{R} produced by the respective arteries of the left and right Inferior limbs and detected by the ankle-pulse-wave detecting devices 44, 46 each functioning as the inferior-limb pulse-wave detecting device; and the phase-difference judging means 68 (S5) that judges whether the phase difference DT_{L}, DT_{R} calculated by the phase-difference calculating means 66 is smaller than the reference value t_{J}. And, it is judged that the artery of at least one of the left and right superior limbs has stenosis, when the phase-difference judging means 68 judges that the phase difference DT_{L}, DT_{R} is smaller than the reference value t_{J}. Thus, the stenosis of each of the left-superior-limb and right-superior-limb arteries can be accurately evaluated.

Also, in the illustrated embodiment, it is judged whether the artery of at least one of the left and right superior limbs has stenosis, based on the phase difference DT_{L}, DT_{R} between each of the left-brachium pulse wave WB_{L} and the right-brachium pulse wave WB_{R} produced by the respective arteries of the left and right superior limbs and detected by the brachium-pulse-wave detecting devices 40, 42 each functioning as the superior-limb pulse-wave detecting device, and one of the left-ankle pulse wave WA_{L} and the right-ankle pulse wave WA_{R} produced by the respective arteries of the left and right inferior limbs and detected by the ankle-pulse-wave detecting devices 44, 46 each functioning as the inferior-limb pulse-wave detecting device, the one ankle pulse wave WA having the faster phase than that of the other ankle pulse wave. If it is assumed that both the left-ankle and right-ankle pulse waves WA_{L}, WA_{R} can be influenced by stenosis, then it can be said that one of the left-ankle and right-ankle pulse waves that has the faster phase is less influenced by stenosis than the other ankle pulse wave. This is the reason why the stenosis of the superior-limb artery can be accurately evaluated.

Also, in the illustrated embodiment, the arteriostenosis judging means 62 judges whether the artery of at least one of the left and right superior limbs has stenosis, based on the phase difference DT_{L}, DT_{R} calculated using the rising point t_{BL} of the left-brachium pulse wave WB_{L}, the rising point t_{BR} of the right-brachium pulse wave WB_{R}, and the rising point t_{A} of the ankle pulse wave WA. Thus, the stenosis of the superior-limb artery can be accurately evaluated. A rising point of a pulse wave is less influenced by a reflected wave than a peak point of the pulse wave. This is the reason why the stenosis of the superior-limb artery can be accurately evaluated.

Also, the illustrated apparatus includes the indication outputting means 70 that outputs, when the arteriostenosis judging means 62 judges that the artery of at least one of the left and right superior limbs has stenosis, the indication indicating that the artery of the superior limb has stenosis. Thus, a person such as the subject or a doctor can assuredly recognize the stenosis of the superior-limb artery.

While the present invention has been described in its embodiment by reference to the drawings, it is to be understood that the invention may otherwise be embodied.

For example, in the illustrated embodiment, the four pulse-wave detecting devices 40, 42, 44, 46 are employed. However, it is needed to employ at least one of the two brachium pulse-wave detecting devices 40, 42 and at least one of the two ankle pulse-wave detecting devices 44, 46.

In addition, the left brachium cuff 20L, the right brachium cuff 20R, the left ankle cuff 18L, and the right ankle cuff 18R of the pulse-wave detecting devices 40, 42, 44, 46 may be worn on other body portions of the living subject than the brachia and the ankles of the subject so as to detect respective pulse waves therefrom. For example, cuffs of pulse-wave detecting devices may be worn on wrists in place of the brachia, or may be worn on articulations of foot, in place of the ankles. Also, cuffs of pulse-wave detecting devices may be worn on femoral portions so as to detect respective pulse waves therefrom.

The pulse-wave detecting devices 40, 42, 44, 46 may be replaced by other sorts of pulse-wave detecting devices, such as a photoelectric-pulse-wave detecting probe for use in oxygen-saturation measurement; a pressure-pulse-wave sensor that is pressed against an appropriate artery, e.g., a radial artery via skin and detects a pressure pulse wave produced from the artery; an impedance-pulse-wave sensor that detects impedance of an arm or a finger via electrodes; or a photoelectric-pulse-wave sensor that is worn on, e.g., a tip of a finger so as to detect pulsation.

The display device 54 displays, as shown in FIG. 4, the left-brachium pulse wave WB_{L}, the right-brachium pulse wave WB_{R}, and the selected one WA of the left and right ankle pulse waves WA_{L}, WA_{R} that has the faster phase, such that the three-pulse waves are arranged in a vertical direction and run parallel to each other. However, it is possible to display the three pulse waves such that at least two of the three pulse waves are superposed on each other.

The display device 54 displays, as shown in FIG. 4, the left-brachium pulse wave WB_{L}, the right-brachium pulse wave WB_{R}, and the selected one WA of the left and right ankle pulse waves WA_{L}, WA_{R} that has the faster phase, such that the three pulse waves are arranged in a vertical direction and run parallel to each other. However, it is possible to display, in place of, or in addition to, the selected one ankle pulse wave WA, both of the left and right ankle pulse waves WA_{L}, WA_{R}.

In the illustrated embodiment, the phase-difference calculating means 66 calculates the phase difference DT_{L} between the ankle pulse wave WA selected by the pulse-wave selecting means 64 and the left-brachium pulse wave WB_{L} detected by the left-brachium pulse wave detecting device 40, and the phase difference DT_{R} between the selected ankle pulse wave WA and the right-brachium pulse wave WB_{R} detected by the right-brachium pulse wave detecting device 42. However, the phase-difference calculating means 66 may be so modified as to calculate a phase difference DT_{L} between a pre-selected one WA of the left-ankle and right-ankle pulse waves WA_{L,} WA_{R} respectively detected by the left-ankle and right-ankle pulse-wave detecting devices 44, 46 and the left-brachium pulse wave WB_{L} detected by the left-brachium pulse wave detecting device 40, and/or a phase difference DT_{R} between the pre-selected ankle pulse wave WA and the right-brachium pulse wave WB_{R} detected by the right-brachium pulse wave detecting device 42. Even the thus modified phase-difference calculating means 66 can enjoy an advantage. Therefore, the pulse-wave selecting means 64 may be omitted.

The phase-difference calculating means 66 calculates the phase difference DT_{L} and the phase difference DT_{R}, by using, as the reference points, the rising point t_{A} of the ankle pulse wave WA, the rising point t_{BL} of the left-brachium pulse waves. WB_{L}, and the rising point t_{BR} of the right-brachium pulse wave WB_{R}. However, the reference points of the pulse waves, may be other points such as maximum-value (i.e., peak) points or greatest-slope points.

While the present invention has been described in its embodiments in detail by reference to the drawings, it may be understood that the present invention is by no means limited to the details of the embodiments but may be embodied with various changes and improvements that may occur to a person skilled in the art.

What is claimed is:

1. A superior-limb arteriostenosis evaluating apparatus, comprising:
a superior-limb pulse-wave detecting device which detects a superior-limb pulse wave produced from an artery of at least one of a left superior limb and a right superior limb of a living subject;
an inferior-limb pulse-wave detecting device which detects an inferior-limb pulse wave produced from an artery of at least one of a left inferior limb and a right inferior limb of the subject; and
an arteriostenosis judging means for judging whether the artery of said at least one of the left and right superior limbs has an arteriostenosis, based on a phase difference between the superior-limb pulse wave detected by the superior-limb pulse-wave detecting device and the inferior-limb pulse wave detected by the inferior-limb pulse-wave detecting device.

2. A superior-limb arteriostenosis evaluating apparatus according to claim 1, wherein the arteriostenosis judging means comprises:
a phase-difference calculating means for calculating the phase difference between the superior-limb pulse wave detected by the superior-limb pulse-wave detecting device and the inferior-limb pulse wave detected by the inferior-limb pulse-wave detecting device; and
a phase-difference judging means for judging whether the phase difference calculated by the phase-difference calculating means is smaller than a reference value, and judging that the artery of said at least one of the left and right superior limbs has said arteriostenosis, when it is judged that the phase difference calculated by the phase-difference calculating means is smaller than the reference value.

3. A superior-limb arteriostenosis evaluating apparatus according to claim 1, wherein the inferior-limb pulse-wave detecting device detects a left-inferior-limb pulse wave produced from an artery of the left inferior limb and a right-inferior-limb pulse wave produced from an artery of the right inferior limb, wherein the arteriostenosis judging means comprises:
a pulse-wave selecting means for selecting one of the left-inferior-limb pulse wave and the right-inferior-limb pulse wave that has a faster phase than a phase of the other inferior-limb pulse wave; and
means for judging whether the artery of said at least one of the left and right superior limbs has said arteriostenosis, based on the phase difference between the superior-limb pulse wave detected by the superior-limb pulse-wave detecting device and said one inferior-limb pulse wave selected by the pulse-wave selecting means.

4. A superior-limb arteriostenosis evaluating apparatus according to claim 1, wherein the arteriostenosis judging means judges whether the artery of said at least one of the left and right superior limbs has said arteriostenois, based on the phase difference between a rising point of the superior-limb pulse wave detected by the superior-limb pulse-wave detecting device and a rising point of the inferior-limb pulse wave detected by the inferior-limb pulse-wave detecting device.

5. A superior-limb arteriostenosis evaluating apparatus according to claim 1, further comprising an indication outputting means for outputting, when the arteriostenosis judging means judges that the artery of said at least one of the left and right superior limbs has said arteriostenosis, an indication indicating that the artery of said at least one of the left and right superior limbs has said arteriostenosis.

6. A superior-limb arteriostenosis evaluating apparatus, comprising:
a superior-limb pulse-wave detecting device which detects a superior-limb pulse wave produced from an artery of at least one of a left superior limb and a right superior limb of a living subject;
an inferior-limb pulse-wave detecting device which detects an inferior-limb pulse wave produced from an artery of at least one of a left inferior limb and a right inferior limb of the subject; and
an arteriostenosis judging device which judges whether the artery of said at least one of the left and right superior limbs has an arteriostenosis, based on a phase difference between the superior-limb pulse wave detected by the superior-limb pulse-wave detecting device and the inferior-limb pulse wave detected by the inferior-limb pulse-wave detecting device.
